(19) United States
(12) Patent Application Publication
(10) Pub. No.: US 2006/0219951 A1
(43) Pub. Date: Oct. 5, 2006

(54) Charged particle beam exposure equipment and charged particle beam exposure method

(76) Inventor: Hideaki Komami, Tokyo (JP)

Correspondence Address: MURAMATSU & ASSOCIATES, Suite 310, 114 Pacifica, Irvine, CA, 92618, US

(21) Appl. No.: 11/352,692

(22) Filed: Feb. 13, 2006

(30) Foreign Application Priority Data
Feb. 15, 2005 (JP) 2005-037183

(51) Int. Cl. A61N 5/00; G21G 5/00

(52) U.S. Cl. 250/492.3

(57) ABSTRACT

A charged particle beam exposure equipment includes an exposure module for exposing a resist film, to which an antistatic film is adhered, by means of irradiating a charged particle beam on the resist film, and a post-exposure wafer processing module for process a post-exposure wafer, which includes antistatic film removing means which detaches the antistatic film after exposure; baking means which causes the wafer to undergo a baking process; and post-exposure wafer process controlling means which transfers the post-exposure wafer to the antistatic film removing means, and which causes the antistatic film removing means to remove the antistatic film from the post-exposure wafer, thereafter transferring the resultant wafer to the baking means.

### CROSS-REFERENCE TO RELATED APPLICATIONS

This application is based on and claims priority of Japanese Patent Application No. 2005-37183 filed on Feb. 15, 2005, the entire contents of which are being incorporated herein by reference.

### BACKGROUND OF THE INVENTION

1. Field of the Invention

The present invention relates to charged particle beam exposure equipment and a charged particle beam exposure method. In particular, the present invention relates to charged particle beam exposure equipment for exposing a resist on which an antistatic film is formed, and to the charged particle beam exposure method.

2. Description of the Prior Art

For the purpose of forming a fine pattern, charged particle beam exposure equipment has become used in the lithography process in manufacture of semiconductor devices in recent years.

While fine patterns are being formed by use of the charged particle beam exposure equipment, the following phenomenon occurs. Electric charge is accumulated while the resist is being used. This charge-up deflects the charged particle beam, and accordingly this deteriorates the writing-position accuracy.

For the purpose of preventing this phenomenon, a conductive antistatic film is generally formed on top of, or on the back of, the resist. By means of forming this antistatic film, the electric potential of the antistatic film is used, for example, as the earth potential. Thus, the resist is not to be electrically charged.

With regard to technologies related to this, Japanese Patent Laid-open Official Gazette No. Hei. 2-5408 has disclosed a method, according to which an antistatic film is formed on the back of a resist used for the patterning process for the purpose of preventing the charge-up. In addition, Japanese Patent Laid-open Official Gazette No. Hei. 7-74076 has disclosed a method, according to which the resist has a two-layered structure. In this two-layered structure, an antistatic film and a fluoride-containing film are laminated.

As described above, in a case where a resist is exposed by use of a charged particle beam, formation of an antistatic film is essential for making sure that the charged particle beam travel straight.

However, if the exposure process and the development process are performed with an antistatic film formed on the resist, the resolution and edge roughness of a pattern to be formed by means of the resist are deteriorated, and accordingly the pattern accuracy is reduce, in some cases, in comparison with a case where the antistatic film is not formed.

### SUMMARY OF THE INVENTION

The present invention has been made in view of such problems with the conventional technologies. An object of the present invention is to provide charged particle beam exposure equipment and a charged particle beam exposure method, which make it possible to form a pattern accurately in a case where an antistatic film is formed on the resist.

The aforementioned problems are solved by charged particle beam exposure equipment which includes an exposure module and a post-exposure wafer processing module. The exposure module performs an exposure process by means of irradiating a charged particle beam on a resist film to which an antistatic film is adhered. The post-exposure wafer processing module processes a post-exposure wafer. The post-exposure wafer processing module includes antistatic film removing means, baking means, and post-exposure wafer process controlling means. The antistatic film removing means removes the antistatic film after exposure. The baking means causes the wafer to undergo a baking process. The post-exposure wafer process controlling means carries the post-exposure wafer to the antistatic film removing means, and carries the wafer to the baking means after the antistatic film is removed.

In addition, the aforementioned problems are solved by a charged particle beam exposure method characterized by including the steps of: exposing, and transferring, a desired pattern to a resist film, on whose surface an antistatic film is formed, by means of a charged particle beam; removing the antistatic film; and performing a baking process of giving the resist film a bake after the antistatic film is removed.

In the case of the present invention, after the wafer is exposed, the antistatic film which has been formed on the resist is removed in the post-exposure wafer processing module. Thereafter, the post exposure bake (PEB) process is performed. This nullifies influence of the antistatic film which has been formed on the resist in the PEB process. Accordingly, this makes it possible to prevent the pattern accuracy from being deteriorated.

Furthermore, in the case of the present invention, the charged particle beam exposure equipment is provided with the post-exposure wafer processing module for performing the antistatic film removing process and the baking process. In addition, immediately after exposure, the antistatic film removing process and the baking process are designed to be performed. This makes it possible to prevent a phenomenon which would otherwise deform the pattern due to the post-exposure wafer being left as it is.

### BRIEF DESCRIPTION OF THE DRAWINGS

FIG. 1 is a diagram of a configuration of charged particle beam equipment used in the case of an embodiment of the present invention.

FIG. 2 is a cross-sectional view for describing a charged particle beam exposure method according to the present invention.

FIG. 3 A is an SEM image (part one) of a pattern which is obtained in a case where an antistatic film is removed, followed by a PEB process and a development process. FIG. 3B is an SEM image (part one) of a pattern which is obtained in a case where the PEB process is performed, followed by removal of the antistatic film and a development process. FIG. 3C is an SEM image (part one) of a pattern which is obtained in a case where a development process is performed with no antistatic film.

FIG. 4 A is an SEM image (part two) of a pattern which is obtained in a case where an antistatic film is removed, followed by a PEB process and a development process. FIG. 4B is an SEM image (part two) of a pattern which is obtained in a case where the PEB process is performed, followed by removal of the antistatic film and a development process. FIG. 4C is an SEM image (part two) of a pattern which is obtained in a case where a development process is performed with no antistatic film.

### DESCRIPTION OF THE PREFERRED EMBODIMENT

Hereinafter, descriptions will be provided for an embodiment of the present invention with reference to the drawings.

To begin with, descriptions will be provided for a configuration of charged particle beam exposure equipment, a configuration of a post-exposure wafer processing module, and a controlling module for controlling each of the components. Subsequently, descriptions will be provided for operations performed by the post-exposure wafer processing module of the charged particle beam exposure equipment which is characteristic of the present invention. Thereafter, descriptions will be provided for an exposure method of enhancing accuracy with which a resist to be adhered to an antistatic film is formed by use of the charged particle beam exposure equipment. Finally, an example will be shown, in which a pattern is formed in a resist accurately by use of the exposure equipment and the exposure method according to the present invention.

(Configuration of the Charged Particle Beam Exposure Equipment)

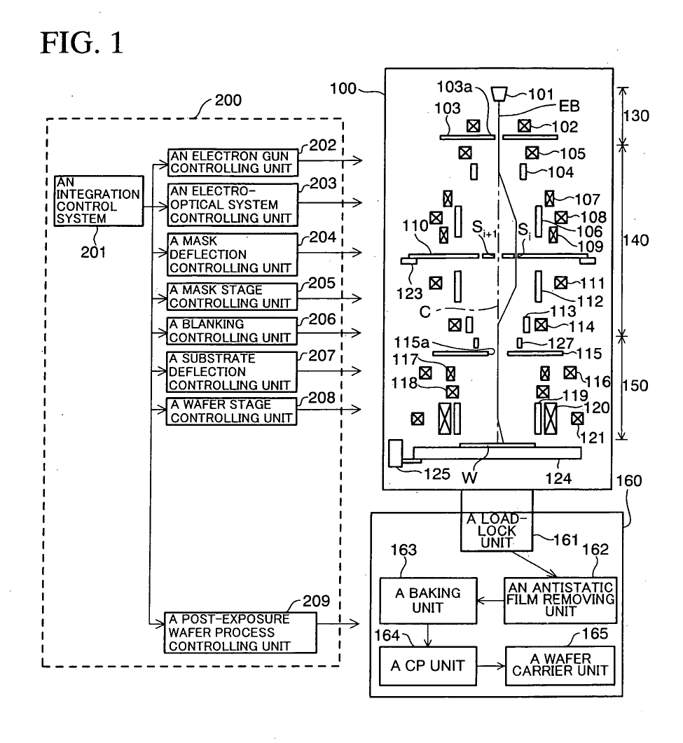

FIG. 1 is a diagram of a configuration of charged particle beam exposure equipment according to the present invention.

This charged particle beam exposure equipment is divided into an exposure module 100, a post-exposure wafer processing module 160, and a control module 200 which controls each of the exposure module 100 and the post-exposure wafer processing module 160. Out of these modules, the exposure module 100 is configured of a charged particle beam generating unit 130, a mask deflection unit 140 and a substrate deflection unit 150, and the pressure inside the exposure module 100 is reduced.

In the charged particle beam generating unit 130, a charged particle beam EB generated by an electron gun 101 is caused to undergo convergence effects in a first electromagnetic lens 102. Thereafter, the charge particle beam EB is transmitted through a rectangular aperture 103a of a beam shaping mask 103, and thereby the cross section of the charged particle beam is shaped into a rectangle.

Subsequently, the charged particle beam EB forms an image on an exposure mask 110 by means of a second electromagnetic lens 105 in a mask deflection unit 140. Then, the charged particle beam EB is deflected onto a specific pattern S formed on the exposure mask 110 by a first and a second electrostatic deflectors 104 and 106, and thus the cross section of the charged particle beam is shaped into a shape of the pattern S.

It should be noted that, although the exposure mask 110 is fixed to a mask stage 123, the mask stage 123 can be displaced in a horizontal plane. In a case where the pattern S existing in a part beyond a deflection range (a beam deflection area) of the first and second electrostatic deflectors 104 and 106 is intended to be used, the pattern S is displaced to the beam deflection area by means of displacing the mask stage 123.

A third and a fourth electromagnetic lenses 108 and 111 arranged respectively above and under the exposure mask 110 play roles in causing the charged particle beam to form the image on a substrate W by means of adjusting amounts of current in the lenses 108 and 111.

The charged particle beam EB which has passed through the exposure mask 110 is swung back to the optical axis C by means of deflection effects of a third and a fourth deflectors 112 and 113. Thereafter, the charged particle beam EB is reduced in size by a fifth electromagnetic lens 114.

A mask deflecting unit 140 is provided with a first and a second correction coils 107 and 109. Aberration of the beam deflection produced by the first to the fourth electrostatic deflectors 104, 106, 112 and 113 is corrected by these correction coils.

Thereafter, the charged particle beam EB passes through an aperture 115a of a shielding plate 115 constituting a substrate deflection unit 150, and is projected onto the substrate W by a first and a second projection electromagnetic lenses 116 and 121. Thereby, an image of the pattern of the exposure mask 110 is transferred to the substrate W with a predetermined reduction ratio, for example, with a ratio of 1/60.

The substrate deflection unit 150 is provided with a fifth electrostatic deflector 119 and an electromagnetic deflector 120. The charged particle beam EB is deflected by these deflectors 119 and 120. Thereby, the image of the pattern of the exposure mask is projected onto a predetermined position in the substrate W.

In addition, the substrate deflection unit 150 is provided with a third and a fourth correction coils 117 and 118 for the purpose of correcting deflection aberration of the charged particle beam EB on the substrate W.

The substrate W is fixed to a wafer stage 124 which can be displaced in the horizontal plane by a drive unit 125 such as a motor. The exposure can be applied to the entire surface of the substrate W by means of displacing the wafer stage 124.

(Configuration of the Post-exposure Wafer Processing Module)

The post-exposure wafer processing module 160 is configured of a load-lock unit 161, an antistatic film removing unit 162, a baking unit 163, a CP (chill plate) unit 164 and a wafer carrier unit 165. The load-lock unit 161 is connected with the exposure module 100. The antistatic film removing module 162 detaches an antistatic film. The baking unit 163 performs a PEB process. The CP unit 164 cools down the wafer. The wafer carrier unit 165 stores the wafer.

(Description of Control Module)

On the other hand, the control module 200 includes an electron gun controlling unit 202, an electro-optical system controlling unit 203, a mask deflection controlling unit 204, a mask stage controlling unit 205, a blanking controlling unit 206, a substrate deflection controlling unit 207, a wafer stage controlling unit 208 and a post-exposure wafer process controlling unit 209. Out of these units, the electron gun controlling unit 202 controls the electron gun 101, and thus controls an acceleration voltage, conditions for the beam radiation of the charged particle beam EB, and the like. In addition, the electro-optical system controlling unit 203 controls an amount of electric current and the like to each of the electromagnetic lenses 102, 105, 108, 111, 114, 116 and 121, and thus adjusts magnifications and focal positions of the electro-optical system in which these electromagnetic lenses are constructed. The blanking controlling unit 206 controls a voltage to be applied to a blanking electrode 127, and thereby deflects the charged particle beam EB, generated before exposure, to the shielding plate 115, thus preventing the charged particle beam EB from being irradiated on the wafer W before the exposure.

The substrate deflection controlling unit 207 controls a voltage applied to the fifth electrostatic deflector 119 and an amount of electric current supplied to the electromagnetic deflector 120, and thereby deflects the charged particle beam EB to the predetermined position on the substrate W. The wafer stage controlling unit 208 displaces the substrate W in the horizontal direction by means of adjusting an amount of drive of the drive unit 125. Thereby, the charged particle beam EB is irradiated on the desired position on the substrate W. The post-exposure wafer process controlling unit 209 causes the wafer to be carried to each unit in the post-exposure wafer processing module 160, adjusts an amount of water which is necessary while the antistatic film is removed, and adjusts the baking temperature.

The aforementioned units 202 to 209 are jointly controlled by an integration control system 201 such as a workstation.

In the case of the charged particle beam exposure equipment configured in the aforementioned manner, the antistatic film removing unit 162, the baking unit 163, the CP unit 164, the wafer carrier unit 165 and the post-exposure wafer process controlling unit 209 correspond respectively to “antistatic film removing means,” “baking means,” “chill means,” “wafer storage means” and “post-exposure wafer process controlling means.”

(Operations of Post-Exposure Wafer Processing Module)

With reference to the drawing, hereinafter, descriptions will be provided for operations of the post-exposure wafer process module 160 which performs processes after the charged particle beam is irradiated on the resist film to which the antistatic film is adhered, followed by exposure.

After the exposure, the wafer is transferred to the post-exposure wafer processing module 160 via the load-lock unit 161. Before the PEB process, the wafer is placed in a cup (a holding platform) in the antistatic film removing unit 162 for the purpose of removing the antistatic film from the wafer. Then, the cup in which the wafer is placed is rotated. Pure water is dropped, or sprayed, onto the surface of the wafer while the wafer is being rotated, and thus the antistatic film is removed (detached) from the wafer. The wafer from which the antistatic film has been detached is transferred to the baking unit 163, and is caused to undergo the PEB process. The wafer which has undergone the PEB process is transferred to the CP unit 164, and the CP unit 164 cools down the wafer, whose temperature has risen due to the PEB process. Subsequently, the wafer is transferred to the wafer carrier unit 165.

In this respect, the antistatic film is made of polyaniline-sulfonic acid (5%) and water (95%), and is water-soluble. For this reason, the antistatic film formed on the resist can be easily detached from the wafer by means of dropping or spraying water to the wafer.

As described above, in the case of this embodiment, after the wafer is exposed, the antistatic film is detached from the wafer in the post-exposure wafer processing module, and thereafter the PEB process is performed. This nullifies influence of the antistatic film formed on the resist during the PEB process, and to accordingly prevent the pattern accuracy from being deteriorated.

In addition, the charged particle beam exposure equipment is provided with the post-exposure wafer processing module for performing the antistatic film removing process and the baking process. Thereby, the antistatic film removing process and the baking process are designed to be performed immediately after the exposure. This makes it possible to prevent a phenomenon which would otherwise deform the pattern due to the post-exposure wafer being left as it is.

(Description of Charged Particle Beam Exposure Method)

Next, descriptions will be provided for a exposure method of accurately forming a pattern on the resist, to which the antistatic film is adhered, by use of the charged particle beam exposure equipment having the aforementioned post-exposure wafer processing module.

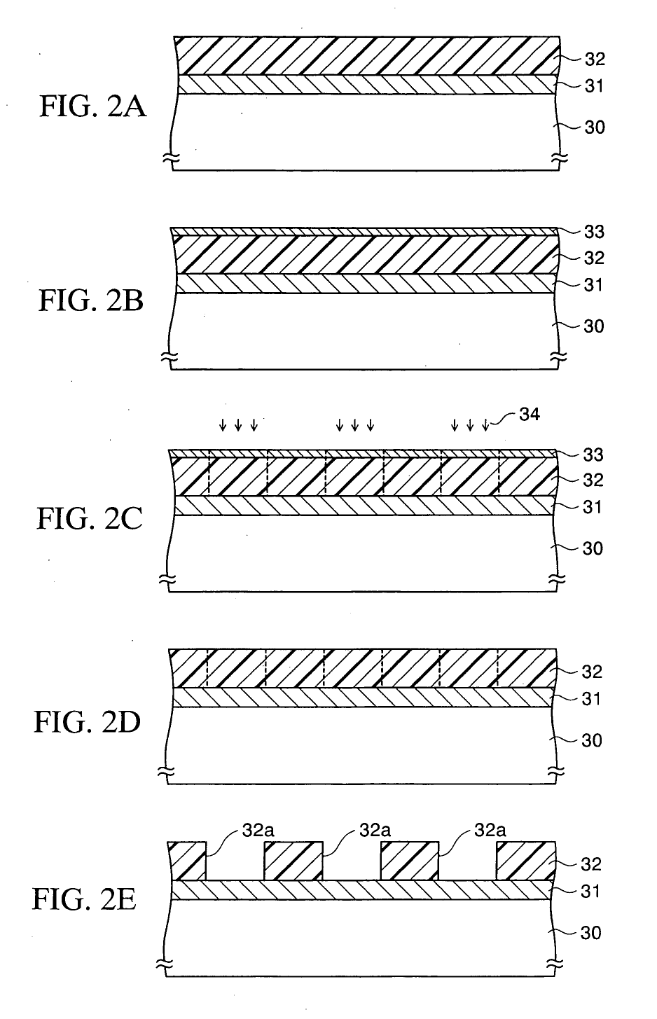

FIGS. 2A to 2E are cross-sectional views sequentially showing steps of the charged particle beam exposure method according to this embodiment for the purpose of describing the method.

In this regard, descriptions will be provided for a workpiece which is obtained by forming an insulating film on a wafer (substrate) and thereafter applying a photosensitive resist to the resultant wafer.

First of all, as shown in FIG. 2A, a silicon oxide film 31 is formed on a wafer (substrate) 30 made of silicon. Subsequently, a resist 32 is applied, to a film thickness of approximately 150 nm, to the resultant wafer by use of the spin coating method. At this point, a chemically-amplified resist is used as the resist 32. The chemically-amplified resist produces an acid in the resist film through a photoreaction, forms the pattern with the acid used as a catalyst while heated after the exposure. In the case of this embodiment, a resist whose sensitivity is 15 μC/cm² is used as the chemically-amplified resist.

Then, as shown in FIG. 2B, the antistatic film 33 is formed on the entire upper surface of the resist 32. A conductive material to be made into the antistatic film 33 is applied, to a film thickness of approximately 30 nm, to the entire upper surface of the resist 32, by the spin coating method. In the case of this embodiment, a conductive material made of polyaniline-sulfonic acid (5%) and water (95%) is used as the antistatic film 33. Thereafter, the baking process is performed for the purpose of dehydration.

Subsequently, a desired pattern is exposed by means of the charged particle beam 34, as shown in FIG. 2C. The exposure is performed while the antistatic film 33 is grounded. This makes it possible to prevent electric charges from being accumulated in the resist 32 and the silicon oxide film 31. This precludes an incident position of the charged particle beam 34 from shifting due to electrification of the resist 32 and the silicon oxide film 31. Furthermore, since the antistatic film 33 is formed with a thickness as small as 30 nm, it is rarely that the charged particle beam 34 are scattered in the conductive antistatic film 33. The charged particle beam 34 travels straightly across the antistatic film 33, and reaches the resist 32.

Thereafter, the antistatic film 33 is removed from the workpiece, as shown in FIG. 2D. The post-exposure workpiece is transferred from the exposure module 100 in vacuum to the post-exposure wafer processing module 160 under an atmospheric pressure via the load-lock unit 161. The antistatic film 33 is removed from the workpiece, which has been transferred to the post-exposure wafer processing module 160, in the antistatic film removing unit 162. The antistatic film 33 is removed in the following manner. First, the workpiece is set in the cup constituting the antistatic film removing unit 162. Then, the cup is rotated. Subsequently, pure water is dropped, or sprayed, to the workpiece from above. Since the antistatic film 33 is water-soluble as described above, the antistatic film 33 can be easily removed from the workpiece by means of dropping, or spraying, pure water to the workpiece from above. In addition, the resist 32 and water do not react on each other. For this reason, use of pure water for removing the antistatic film 33 therefrom does not affect the resist 32.

It should be noted that it takes several minutes to remove the antistatic film 33 therefrom. For this reason, it is several minutes that are spent before the PEB process, in the case of this embodiment. Accordingly, it is rarely that the pattern formed in the resist 32 is changed, whereas, in some cases, after exposure the pattern is changed if it is left, as it is, for several hours.

Subsequently, the PEB process is performed in the baking unit 163. The PEB process is performed at 100 to 130° C. for 90 to 120 seconds. This PEB process reacts the resin in the chemically-amplified resist 32 to the heat, and thus the pattern is obtained. Since the antistatic film does not excessively react to the heat, it is possible to reduce error in the obtained pattern.

Thereafter, the wafer is transferred to the CP unit 164, and is cooled down. The wafer thus cooled is stored in the wafer carrier unit 165.

Next, the wafer is transferred to a developer (not illustrated), and is caused to undergo a development process. A pattern as shown in FIG. 2E is formed.

### EXAMPLE

Hereinafter, descriptions will be provided for a result of actually forming a pattern on a workpiece by use of the charged particle beam exposure method according to this embodiment. In this respect, FEPS-127B made by Fujifilm Electronic Materials Co., Ltd. was used as the chemically-amplified resist. The film thickness was 150 nm. A pattern to be formed was a line-and-space pattern. The width of the pattern was 80 nm, and a ratio of the line width to the space width was 1:2.

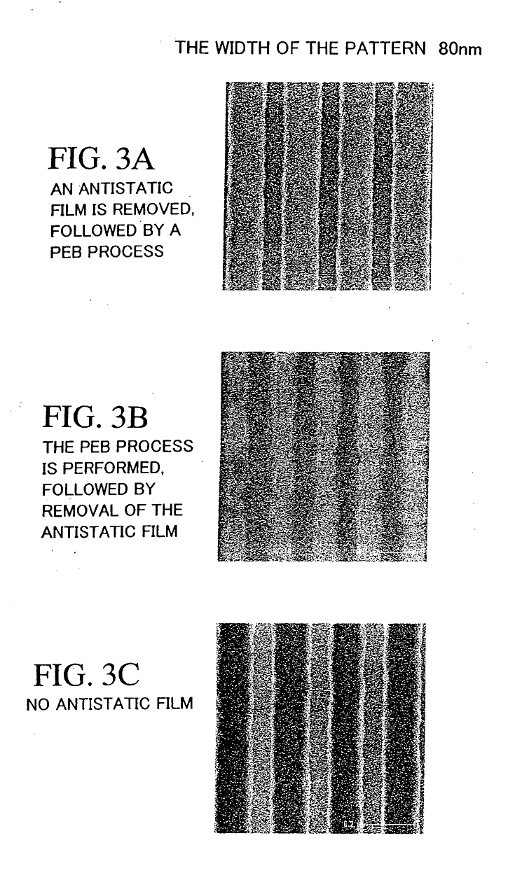

FIG. 3A is an SEM (Scanning Electron Microscope) image of a pattern which was obtained by removing an antistatic film after exposure followed by the PEB process, in a case where the antistatic film was present. FIG. 3B is an SEM image of a pattern which was obtained by performing the PEB process after exposure, and by thereafter removing the antistatic film, in a case where the antistatic film was present. Moreover, FIG. 3C is an SEM image of a pattern which has obtained in a case where the antistatic film was not present.

As learned from FIG. 3A, a pattern with as good a resolution as that of a pattern which was obtained in the case, as shown in FIG. 3C where the antistatic film was not present was obtained in the case where the antistatic film was removed followed by performing the PEB process. On the other hand, in the case where the PEB process was performed followed by removal of the antistatic film, the resolution of the pattern was poor, and the desired pattern was unable to be obtained. Incidentally, in a case where the PEB process was performed with a 1:1 ratio of the line width to the space width followed by removal of the antistatic film, individual parts of the pattern were not made distinguishable, and accordingly the pattern was unable to be obtained.

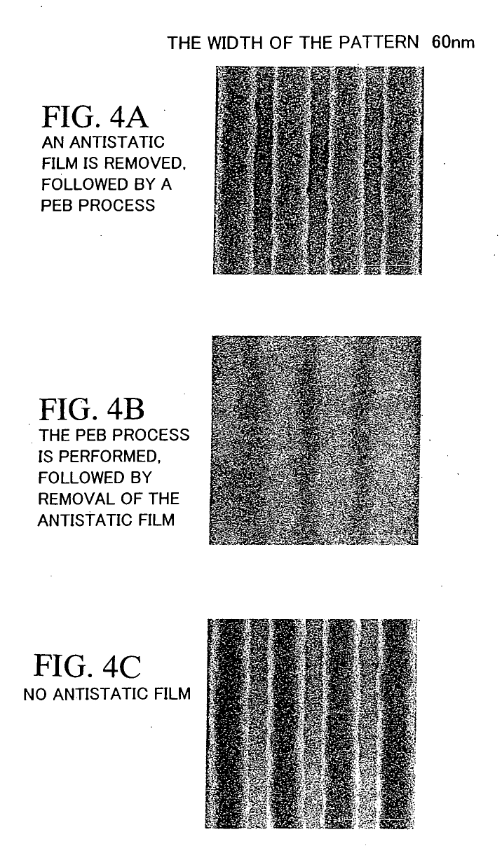

FIGS. 4A to 4C are SEM images of patterns which were obtained by case of a pattern of 60 nm in width and with a 1:2 ratio of the line width to the space width. FIG. 4A is an SEM image of a pattern which was obtained in the case where the antistatic film was removed after exposure, and thereafter the PEB process was performed. FIG. 4B is an SEM image of a pattern which was obtained in the case where the PEB was performed after exposure, and thereafter the antistatic film was removed. FIG. 4C is an SEM image of a pattern which was obtained in the case where no antistatic film was present.

It was confirmed that, in the case where the antistatic film was removed after exposure and thereafter the PEB process was performed, the pattern with a better resolution was able to be obtained.

As described above, in the case of the charged particle beam exposure equipment according to the present invention, an antistatic film is formed on a chemically-amplified resist, and thereafter a desired pattern is exposed. Subsequently, the antistatic film is removed, and then the PEB process is performed. Accordingly, this makes it possible to nullify influence of the antistatic film on the resist in the PEB process, and to increase the pattern accuracy.

In addition, the antistatic film is designed to be removed, and the PEB process is designed to be performed, in the post-exposure wafer processing module which is provided to the exposure equipment so that the module is a part of exposure equipment. For this reason, the PEB process can be applied to the wafer within a short time after exposure. Accordingly, this makes it possible to perform the PEB process before the pattern changes in shape due to the pattern being left, as it is, after exposure, and to thus prevent the pattern from changing.

The followings should be noted. The post-exposure wafer processing module, which is a part of the charged particle beam exposure equipment described with regard to the present invention, can be separated from the charged particle beam exposure equipment. Thereby, the post-exposure wafer processing module thus separated can be used as an independent post-exposure wafer processing machine while connected to the exposure module of the charged particle exposure equipment.

1. Charged particle beam exposure equipment including an exposure module for exposing a resist film, to which an antistatic film is adhered, by irradiating a charged particle beam on the resist film, and a post-exposure wafer processing module for processing a post-exposure wafer, wherein the post-exposure wafer processing module comprises:
antistatic film removing means which detaches the antistatic film after exposure;
baking means which causes the wafer to undergo a baking process; and
post-exposure wafer process controlling means which transfers the post-exposure wafer to the antistatic film removing means, and which causes the antistatic film removing means to remove the antistatic film from the post-exposure wafer, thereafter transferring the resultant wafer to the baking means.

2. The charged particle beam exposure equipment according to claim 1, wherein the antistatic film removing means comprises:
a holding platform for holding the post-exposure wafer on it; and
any one of dropping means which drops pure water onto the wafer and spraying means which sprays pure water onto the wafer.

3. The charged particle beam exposure equipment according to claim 1, wherein the post-exposure wafer processing module is connected with the exposure module with a load-lock interposed in between.

4. The charged particle beam exposure equipment according to claim 1, wherein the post-exposure wafer processing module further comprises cooling means which cools down the wafer which has been baked in the baking means.

5. The charged particle beam exposure equipment according to claim 1, wherein the post-exposure wafer processing module further comprises wafer storing means which stores the wafer which has been cooled down in the cooling means.

6. A post-exposure wafer processing module comprising:
antistatic film removing means which detaches an antistatic film which is adhered to a resist film;
baking means which causes a wafer to undergo a baking process; and
post-exposure wafer process controlling means which transfers the post-exposure wafer to the antistatic film removing means, and which causes the antistatic film removing means to remove the antistatic film from the post-exposure wafer, thereafter transferring the resultant wafer to the baking means.

7. The post-exposure wafer processing module according to claim 6, wherein the antistatic film removing means comprises:
a holding platform for holding the post-exposure wafer on it; and
any one of dropping means which drops pure water onto the wafer and spraying means which sprays pure water onto the wafer.

8. The post-exposure wafer processing module according to claim 6, further comprising cooling means which cools down the wafer which has been baked in the baking means.

9. The post-exposure wafer processing module according to claim 6, further comprising wafer storing means which stores the wafer which has been cooled down in the cooling means.

10. A charged particle beam exposure method, comprising the steps of:
exposing a resist film, on whose front surface an antistatic film has been formed, by means of irradiating a charged particle beam the resist film, and forming a desired pattern on the resist film;
removing the antistatic film; and
performing a baking process of heating the resist film after the antistatic film is removed.

11. The charged particle beam exposure method according to claim 10, wherein the antistatic film is formed through:
a step of applying a conductive material to the entire front surface of the resist film; and
a step of performing a pre-exposure baking process of heating the resist film to which the material has been applied.

12. The charged particle beam exposure method according to claim 10, wherein the conductive material is made of polyaniline-sulfonic acid and water.

13. The charged particle beam exposure method according to claim 10, wherein the resist film is a chemically-amplified resist film which produces an acid by means of irradiating a charged particle beam.
